(19) United States
(12) Patent Application Publication
(10) Pub. No.: US 2024/0186005 A1
(43) Pub. Date: Jun. 6, 2024

(54) APPARATUS AND METHOD FOR PREDICTING HYPOTENSION BASED ON ARTERIAL PRESSURE WAVELET TRANSFORMATION, AND METHOD FOR TRAINING HYPOTENSION PREDICTION MODEL

(71) Applicants: THE ASAN FOUNDATION, Seoul (KR); NATIONAL CANCER CENTER, Goyang-si (KR); UNIVERSITY OF ULSAN FOUNDATION FOR INDUSTRY COOPERATION, Ulsan (KR)

(72) Inventors: Sunghoon KIM, Seoul (KR); Yongseok PARK, Seoul (KR); Junetae KIM, Goyang-si (KR); Eugene HWANG, Seoul (KR)

(73) Assignees: THE ASAN FOUNDATION, Seoul (KR); NATIONAL CANCER CENTER, Goyang-si (KR); UNIVERSITY OF ULSAN FOUNDATION FOR INDUSTRY COOPERATION, Ulsan (KR)

(21) Appl. No.: 18/438,563

(22) Filed: Feb. 12, 2024

Related U.S. Application Data
(63) Continuation of application No. PCT/KR2022/012121, filed on Aug. 12, 2022.

(30) Foreign Application Priority Data
Aug. 13, 2021 (KR) 10-2021-0107379

(51) Int. Cl. G16H 50/20

(52) U.S. Cl. CPC G16H 50/20

(57) ABSTRACT

There is provided an apparatus for predicting hypotension of a subject. The apparatus comprises a memory configured to store one or more instructions and a pre-trained hypotension prediction model; and a processor configured to execute the one or more instructions stored in the memory, wherein the instructions, when executed by the processor, cause the processor to: determine an arterial blood pressure data of the subject, input the arterial blood pressure data of the subject into the hypotension prediction model, and determine whether the subject has hypotension using an output result of the hypotension prediction model.

### TECHNICAL FIELD

The present disclosure relates to an arterial pressure wavelet transform-based apparatus and method for predicting hypotension, and method for training a hypotension prediction model.

### BACKGROUND

Hypotension during surgery occurs when several causes, such as lack of effective blood volume due to bleeding, decreased myocardial contractility due to cardiovascular disease or anesthetics, and decreased vascular resistance due to sepsis or other drugs, act alone or in combination. Among these, lack of effective blood volume due to bleeding or dehydration in a patient under general anesthesia is considered to be the most important cause of hypotension during surgery.

However, conventional hypotension prediction methods predict hypotension by defining specific features or monitoring raw arterial blood pressure (ABP) values, and thus have a problem that it is impossible to reflect trends in blood pressure changes which are the most important information directly related to the cause of hypotension.

### SUMMARY

An object of the present disclosure is to determine hypotension on the basis of changes in each measurement interval of compressed arterial blood pressure (ABP) data containing information on trends in blood pressure changes.

The aspects of the present disclosure are not limited to the foregoing, and other aspects not mentioned herein will be clearly understood by those skilled in the art from the following description.

In accordance with an aspect of the present disclosure, there is provided an apparatus for predicting hypotension of a subject, the apparatus comprises: a memory configured to store one or more instructions and a pre-trained hypotension prediction model; and a processor configured to execute the one or more instructions stored in the memory, wherein the instructions, when executed by the processor, cause the processor to: determine an arterial blood pressure data of the subject, input the arterial blood pressure data of the subject into the hypotension prediction model, and determine whether the subject has hypotension using an output result of the hypotension prediction model, wherein the hypotension prediction model is pre-trained to determine whether a training subject has hypotension for a training arterial blood pressure data of the training subject.

Herein, the hypotension prediction model may be pre-trained by a training input data including a plurality of intervals of the training arterial blood pressure data of the training subject and a label data including whether or not hypotension has occurred for each of the plurality of intervals of the training arterial blood pressure data.

Herein, the hypotension prediction model may include a first layer trained to extract trend data for each of the plurality of intervals of the training arterial blood pressure data of the training subject by a wavelet-transforming the plurality of intervals of the training arterial blood pressure data of the training subject; and a shapelet data generation module configured to generate a shapelet data corresponding to the trend data.

Herein, the hypotension prediction model may be pre-trained to low-pass filter the training arterial blood pressure data using training parameters trained in the first layer.

Herein, the hypotension prediction model may include a second layer trained to assign a weight to at least one of intervals among the plurality of intervals of the training arterial blood pressure data of the training subject; and a third layer trained to calculate similarity feature value between shapelet data of the at least one interval and the trend data on the basis of the assigned weight.

Herein, the processor may be configured to input the arterial blood pressure data of the subject to the hypotension prediction model, and to calculate similarity between trend data for each of the plurality of intervals of the arterial blood pressure data of the subject and the shapelet data generated by the shapelet data generation module.

Herein, the processor may be configured to calculate hypotension probability for the calculated similarity of the trend data for each of the plurality of intervals of the arterial blood pressure data of the subject using a logistic regression layer.

In accordance with another aspect of the present disclosure, there is provided a method for training a hypotension prediction model performed by an electric device including a processor, the method comprises: preparing a training data including a training input data and a label data, wherein the training input data includes a plurality of intervals of the training arterial blood pressure data of a training subject and, the label data includes whether or not hypotension has occurred for each of the plurality of intervals of the training arterial blood pressure data of the training subject; inputting the training input data into the hypotension prediction model; training the hypotension prediction model to extract trend data for each of the plurality of intervals of the training arterial blood pressure data of the training subject by wavelet-transforming the plurality of intervals of the training arterial blood pressure data of the training subject; and generating a shapelet data corresponding to the trend data.

Herein, the training of the hypotension prediction model to extract trend data may include updating training parameters trained in a first layer such that the hypotension prediction model low-pass filters the training arterial blood pressure data.

Herein, the method for training the hypotension prediction model performed may include training the hypotension prediction model to assign a weight to at least one of intervals among the plurality of intervals of the training arterial blood pressure data of the training subject; and training the hypotension prediction model to calculate similarity feature values between shapelet data of the at least one interval and the trend data on the basis of the assigned weight.

In accordance with another aspect of the present disclosure, there is provided a method for predicting hypotension performed by an apparatus for predicting hypotension of a subject, the method comprises: preparing a pre-trained hypotension prediction model; determining an arterial blood pressure data of the subject; inputting the arterial blood pressure data of the subject into the hypotension prediction model, and determining whether the subject has hypotension as an output result of a hypotension prediction model, wherein the hypotension prediction model is pre-trained to determine whether a training subject has hypotension for a training arterial blood pressure data of the training subject.

In accordance with another aspect of the present disclosure, there is provided a non-transitory computer-readable recording medium storing a computer program, which comprises instructions for a processor to perform a method for predicting hypotension performed by an apparatus for predicting hypotension of a subject, the method comprise: preparing a pre-trained hypotension prediction model; determining an arterial blood pressure data of the subject; inputting the arterial blood pressure data of the subject into the hypotension prediction model, and determining whether the subject has hypotension as an output result of a hypotension prediction model, wherein the hypotension prediction model is pre-trained to determine whether a training subject has hypotension for a training arterial blood pressure data of the training subject.

In accordance with another aspect of the present disclosure, there is provided a non-transitory computer-readable recording medium storing a computer program, which comprises instructions for a processor to perform a method for training a hypotension prediction model performed by an electric device including a processor, the method comprise: preparing a training data including a training input data and a label data, wherein the training input data includes a plurality of intervals of the training arterial blood pressure data of a training subject and, the label data includes whether or not hypotension has occurred for each of the plurality of intervals of the training arterial blood pressure data of the training subject; inputting the training input data into the hypotension prediction model; training the hypotension prediction model to extract trend data for each of the plurality of intervals of the training arterial blood pressure data of the training subject by wavelet-transforming the plurality of intervals of the training arterial blood pressure data of the training subject; and generating a shapelet data corresponding to the trend data.

As described above, according to embodiments of the present disclosure, it is possible to determine hypotension using compressed arterial blood pressure (ABP) data containing information on trends in overall change in blood pressure.

In addition, it is possible to determine hypotension on the basis of changes in each measurement interval of compressed ABP data containing information on trends in overall change in blood pressure to provide appropriate treatment by comparing the changes with general forms related to occurrence of hypotension during surgery.

The effects described in the following specification and potential effects expected by the technical features of the present disclosure are treated as if described in the specification of the present disclosure even if the effects are not explicitly mentioned here.

### BRIEF DESCRIPTION OF THE DRAWINGS

FIG. 1 is a diagram showing an apparatus for predicting hypotension according to an embodiment of the present disclosure.

FIG. 2 is a flowchart showing a method for predicting hypotension according to an embodiment of the present disclosure.

FIG. 3 is a flowchart showing a hypotension prediction model training method according to an embodiment of the present disclosure.

FIGS. 4 to 7 are diagrams illustrating exemplary structures of a hypotension prediction model according to an embodiment of the present disclosure.

FIGS. 8 and 9 are diagrams illustrating a process of performing a wavelet transform on arterial blood pressure (ABP) data for training according to an embodiment of the present disclosure.

FIG. 10 is a diagram illustrating a process of generating shapelet data according to an embodiment of the present disclosure.

FIG. 11 is a diagram illustrating a training process for assigning a weight to a training interval required for hypotension prediction according to an embodiment of the present disclosure.

FIG. 12 is a diagram illustrating a training process for calculating similarity feature values between shape data of a training interval and trend data according to an embodiment of the present disclosure.

FIG. 13 is a diagram showing final selection of statistically significant shapelets among shapelets learned in the method for predicting hypotension according to an embodiment of the present disclosure through logistic regression coefficient analysis.

FIG. 14 is a diagram illustrating searching for samples having, as a maximum similarity, each of shapelets selected in the method for predicting hypotension according to an embodiment of the present disclosure, and totaling the number of samples in each of a case of hypotension and a case of non-hypotension.

FIG. 15 is a graph showing an aggregate for each weighted point in the entire compressed interval in the method for predicting hypotension according to an embodiment of the present disclosure.

FIGS. 16A to 16C are graphs showing visualization of shapelets selected using the method for predicting hypotension according to an embodiment of the present disclosure.

### DETAILED DESCRIPTION

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

Terms used in the present specification will be briefly described, and the present disclosure will be described in detail.

In terms used in the present disclosure, general terms currently as widely used as possible while considering functions in the present disclosure are used. However, the terms may vary according to the intention or precedent of a technician working in the field, the emergence of new technologies, and the like. In addition, in certain cases, there are terms arbitrarily selected by the applicant, and in this case, the meaning of the terms will be described in detail in the description of the corresponding invention. Therefore, the terms used in the present disclosure should be defined based on the meaning of the terms and the overall contents of the present disclosure, not just the name of the terms.

When it is described that a part in the overall specification “includes” a certain component, this means that other components may be further included instead of excluding other components unless specifically stated to the contrary.

In addition, a term such as a “unit” or a “portion” used in the specification means a software component or a hardware component such as FPGA or ASIC, and the “unit” or the “portion” performs a certain role. However, the “unit” or the “portion” is not limited to software or hardware. The “portion” or the “unit” may be configured to be in an addressable storage medium, or may be configured to reproduce one or more processors. Thus, as an example, the “unit” or the “portion” includes components (such as software components, object-oriented software components, class components, and task components), processes, functions, properties, procedures, subroutines, segments of program code, drivers, firmware, microcode, circuits, data, database, data structures, tables, arrays, and variables. The functions provided in the components and “unit” may be combined into a smaller number of components and “units” or may be further divided into additional components and “units”.

Hereinafter, the embodiment of the present disclosure will be described in detail with reference to the accompanying drawings so that those of ordinary skill in the art may easily implement the present disclosure. In the drawings, portions not related to the description are omitted in order to clearly describe the present disclosure.

The present disclosure relates to an arterial pressure wavelet transform-based apparatus and method for predicting hypotension, and a method for training a hypotension prediction model thereof.

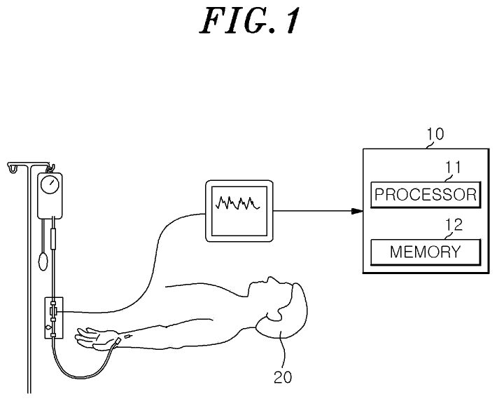

FIG. 1 is a diagram showing an apparatus for predicting hypotension according to an embodiment of the present disclosure.

Referring to FIG. 1, the apparatus 10 for predicting hypotension according to an embodiment of the present disclosure includes a processor 11 and a memory 12.

The apparatus 10 for predicting hypotension according to an embodiment of the present disclosure is an apparatus for predicting whether a subject has hypotension using the arterial blood pressure (ABP) of the subject as a hypotension monitoring index.

As illustrated in FIG. 1, the apparatus 10 for predicting hypotension may receive arterial blood pressure data of a subject 20 during surgery and determine whether the subject has hypotension on the basis of changes in each measurement interval of the arterial blood pressure data.

The apparatus 10 for predicting hypotension according to an embodiment of the present disclosure uses a pre-trained hypotension prediction model and can contribute to improvement of patient prognosis by warning the occurrence of hypotension and providing an efficient decision-making process.

The memory 12 can store programs (one or more instructions) for processing and control of the processor 11, store a pre-trained hypotension prediction model, and include at least one type of computer-readable storage medium among flash memory type, hard disk type, multimedia card micro type, card type memory (for example, SD or XD memory, etc.), random access memory (RAM), static random access memory (SRAM), read-only memory (ROM), electrically erasable programmable read-only memory (EEPROM), programmable read-only memory (PROM), magnetic memory, magnetic disk, and optical disk.

Programs stored in the memory 12 may be divided into a plurality of modules depending on functions, and a pre-trained hypotension prediction model, which will be described later, may be configured as a software module.

The processor 11 executes one or more instructions stored in the memory 12. Specifically, the processor 11 receives arterial blood pressure data of a subject and determines whether the subject has hypotension on the basis of changes in each measurement interval of the arterial blood pressure data using a hypotension prediction model.

In one embodiment of the present disclosure, changes in each measurement interval of arterial blood pressure data mean itemized trend of overall change in blood pressure.

Overall change in blood pressure is the most important information directly related to the cause of hypotension.

Therefore, when low frequency components of compressed arterial blood pressure data containing information of such a trend are extracted and divided into local units, and a trend in each interval is used for prediction, hypotension can be predicted through overall change in local forms.

Here, the processor 11 may be divided into a plurality of modules depending on functions, or functions may be performed by a single processor. The processor may include one or more of a central processing unit (CPU), an application processor (AP), a micro controller unit (MCU), and a communication processor (CP).

The hypotension prediction model of the apparatus 10 for predicting hypotension according to an embodiment of the present disclosure is trained using arterial blood pressure data for training as training data and using whether or not hypotension has occurred in each of a plurality of intervals of the arterial blood pressure data for training as a label.

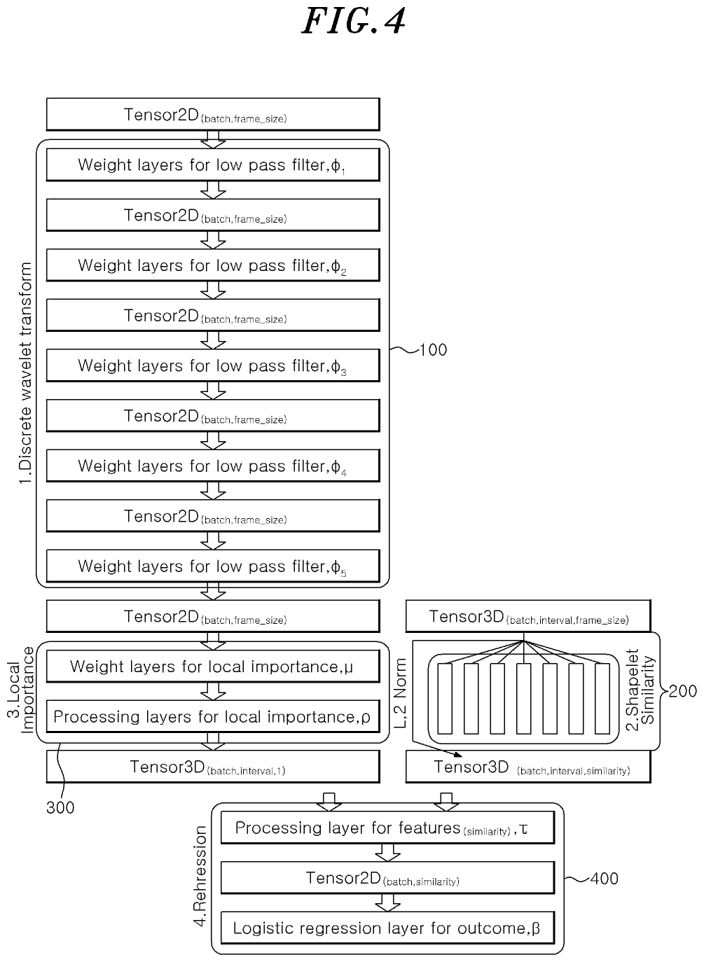

The hypotension prediction model according to an embodiment of the present disclosure will be described in detail using FIGS. 4 to 7.

The processor 11 inputs arterial blood pressure data of a subject to the hypotension prediction model to calculate a similarity between trend data in each measurement interval of the arterial blood pressure data and learned shapelet data.

Thereafter, the probability of occurrence of hypotension is calculated using a logistic regression layer for the calculated similarity of the trend data in each measurement interval, and hypotension is predicted on the basis of the probability of occurrence of hypotension.

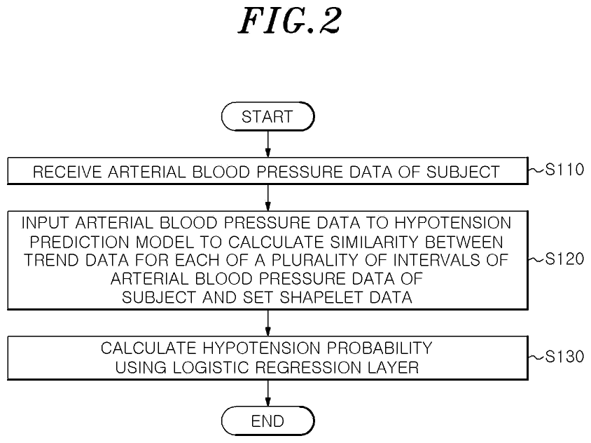

FIG. 2 is a flowchart showing a method for predicting hypotension according to an embodiment of the present disclosure.

Referring to FIG. 2, the method for predicting hypotension according to an embodiment of the present disclosure is performed by the apparatus for predicting hypotension of a subject and includes step S110 in which arterial blood pressure data of a subject is input, and steps S120 and S130 in which it is determined whether the subject has hypotension on the basis of changes in each measurement interval of the arterial blood pressure data using a hypotension prediction model.

Here, the hypotension prediction model is trained using arterial blood pressure data for training as training data and using whether or not hypotension has occurred in each of a plurality of intervals of the arterial blood pressure data for training as a label.

Specifically, in step S120, arterial blood pressure data of the subject is input to the hypotension prediction model to calculate a similarity between trend data in each measurement interval of the arterial blood pressure data and learned shapelet data.

Here, when the arterial blood pressure data is input into the hypotension prediction model, the arterial blood pressure data is optimally compressed in the hypotension prediction model and trend data is extracted.

Thereafter, in step S130, the probability of occurrence of hypotension is calculated using a logistic regression layer for the calculated similarity of the trend data in each measurement interval.

Specifically, trend data is extracted while compressing the arterial blood pressure data, and intervals are divided by overlapping using a sliding method. Thereafter, a similarity between extracted trend data for each interval and shapelet data is calculated, and only the maximum similarity for each shapelet is selected.

For example, if there are 30 shapelets, only 30 similarities remain. Thereafter, the probability is calculated through a logistic regression layer.

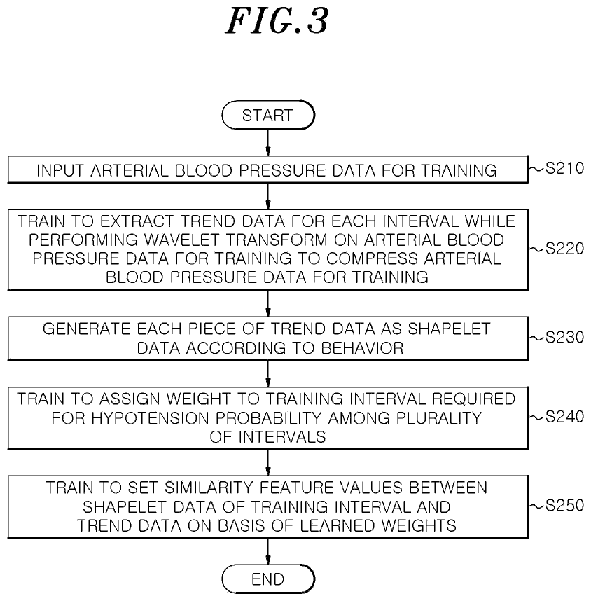

FIG. 3 is a flowchart showing a hypotension prediction model training method according to an embodiment of the present disclosure.

Referring to FIG. 3, the hypotension prediction model training method according to an embodiment of the present disclosure is performed by a processor, and arterial blood pressure data for training is input in step S210.

Thereafter, in step S220, the hypotension prediction model is trained to perform a wavelet transform on the arterial blood pressure data for training to compress the arterial blood pressure data for training and to extract trend data for each interval.

In the process of training the hypotension prediction model to extract trend data, training parameters learned in a first layer are updated such that the arterial blood pressure data for training is low-pass filtered.

Thereafter, in step S230, each piece of trend data is generated as shapelet data according to behavior.

In step S240, the hypotension prediction model is trained to assign a weight to a training interval required for hypotension prediction among a plurality of intervals.

Specifically, a specific interval of the compressed arterial blood pressure data is given a weight and learned to make better prediction.

In step S250, the hypotension prediction model is trained to calculate similarity feature values between shapelet data of the training interval and the trend data on the basis of learned weights.

Here, the hypotension prediction model is caused to learn similarity feature values between the shapelet data and trend data extracted while being compressed.

FIGS. 4 to 7 are diagrams illustrating exemplary structures of the hypotension prediction model according to an embodiment of the present disclosure.

FIG. 4 shows the overall structure of the hypotension prediction model according to an embodiment of the present disclosure.

The hypotension prediction model according to an embodiment of the present disclosure is designed to enable visualization of trend characteristics in local units of arterial blood pressure data within the model.

A prediction model that converts entire time series into compressed behaviors such that trend changes can be checked during surgery and gives explanatory power is generated.

Referring to FIG. 4, the hypotension prediction model according to an embodiment of the present disclosure includes a first layer 100, a shapelet data generation module 200, a second layer 300, and a third layer 400.

In one embodiment of the present disclosure, a layer means a training module including at least one layer, and a layer may collectively refer to a function of a known mathematical structure having reusable and trainable variables.

The first layer 100 is trained to extract trend data for each interval while compressing arterial blood pressure data for training by performing a wavelet transform on the arterial blood pressure data for training.

Here, training is performed to low-pass filter the arterial blood pressure data for training using training parameters learned in the first layer 100.

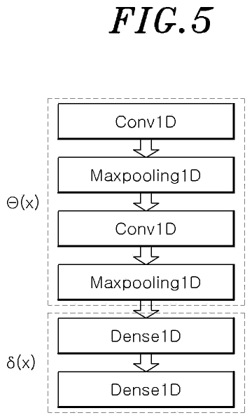

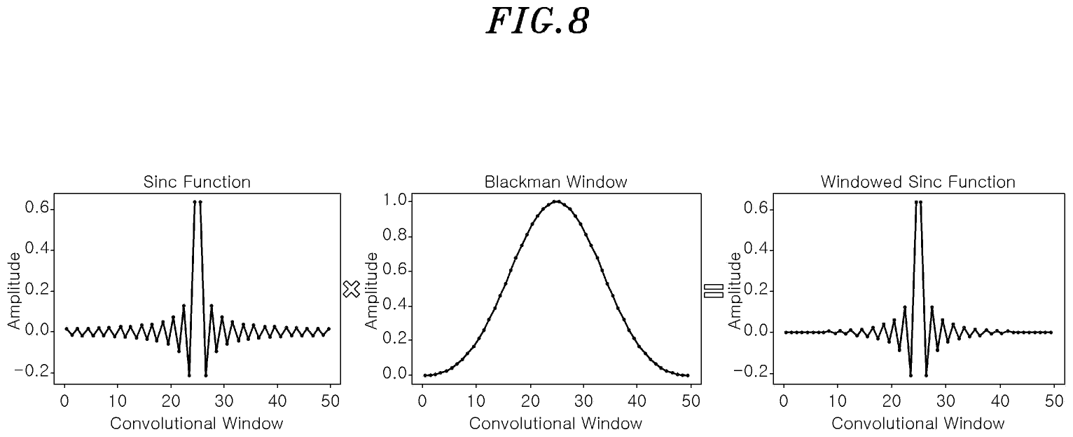

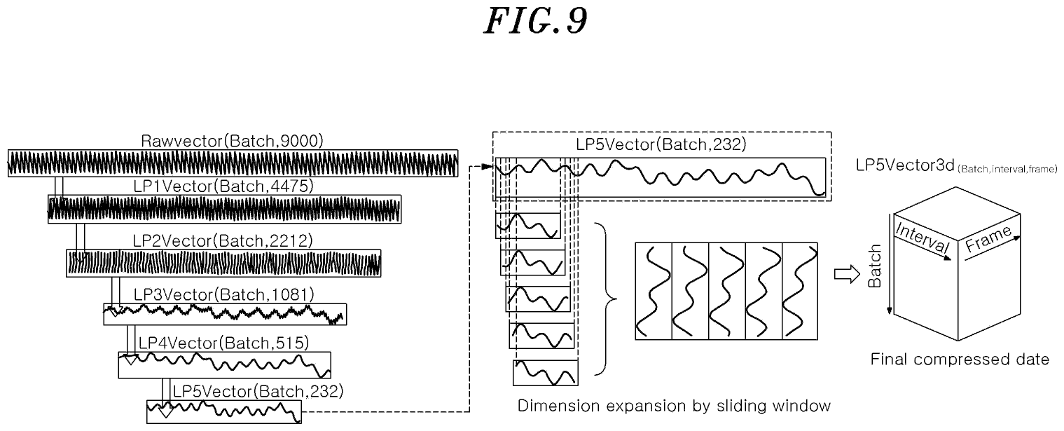

The detailed structure of the first layer 100 will be described in detail with reference to FIG. 5, and the low-pass filtering operation of the first layer 100 for the arterial blood pressure data for training will be described in detail with reference to FIGS. 8 and 9.

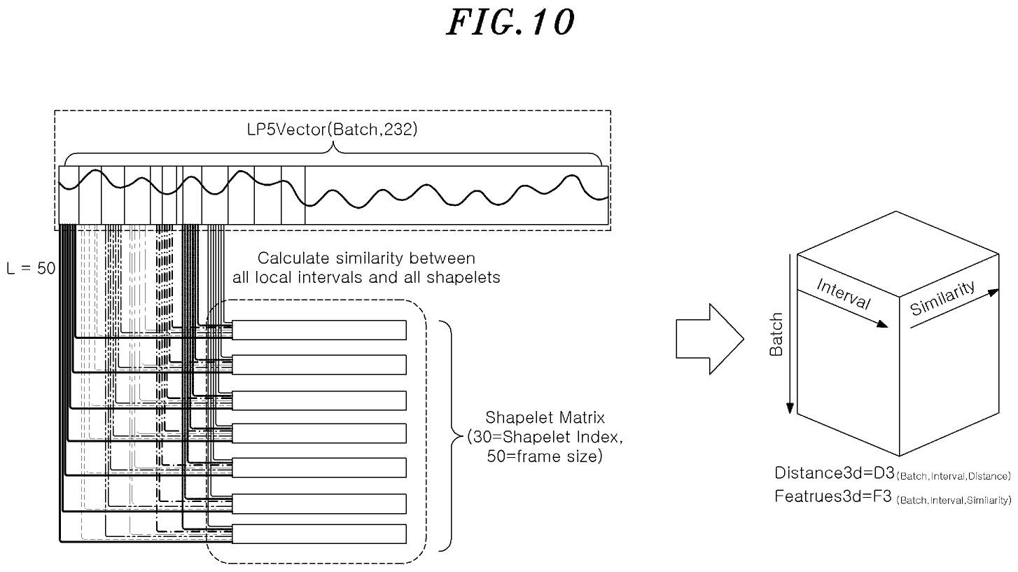

The shapelet data generation module 200 generates each piece of trend data as shapelet data according to behavior. The operation of generating shapelet data will be described in detail with reference to FIG. 10.

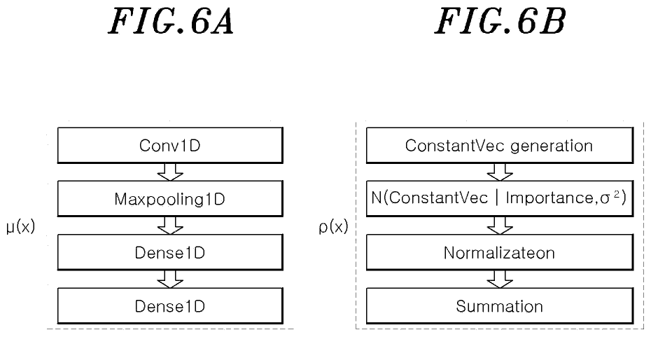

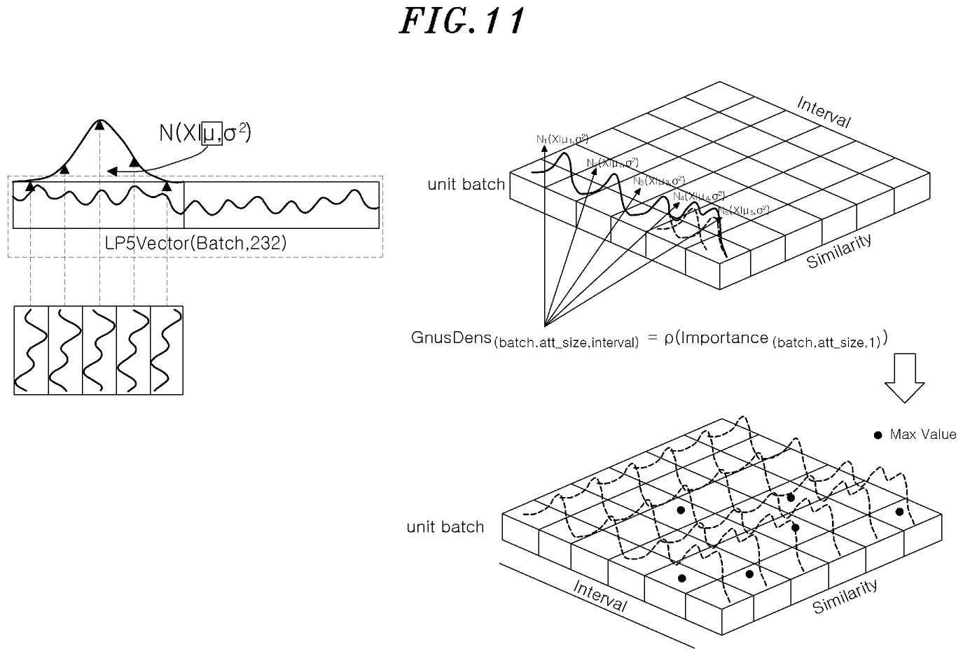

The second layer 300 is trained to assign a weight to a training interval required for hypotension prediction among a plurality of intervals. The detailed structure of the second layer 300 will be described in detail with reference to FIGS. 6A and 6B, and the weight processing operation of the second layer 300 will be described in detail with reference to FIG. 11.

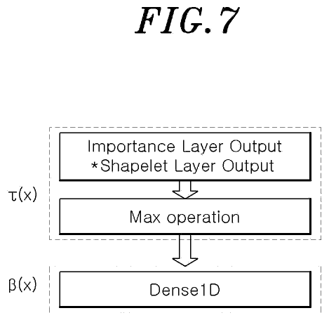

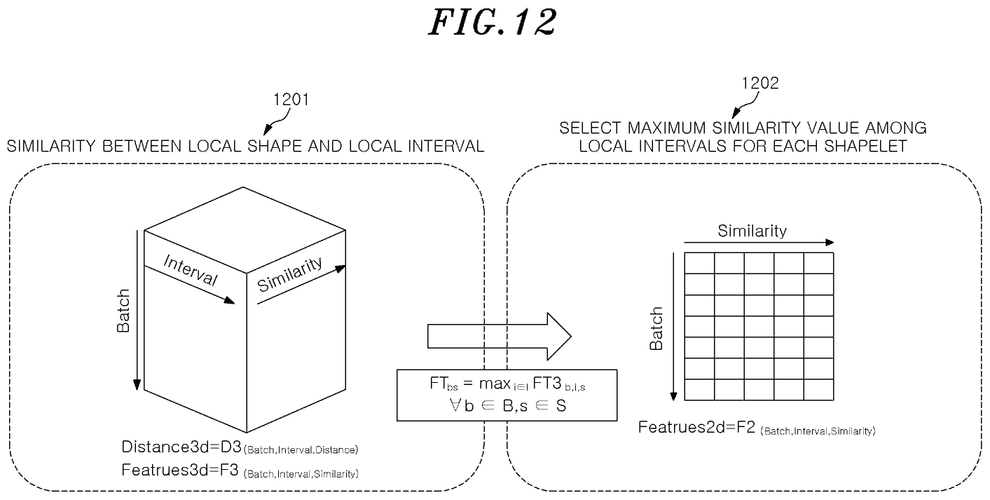

The third layer 400 is trained to calculate similarity feature values between shapelet data of the training interval and the trend data on the basis of learned weights. The detailed structure of the third layer 400 will be described with reference to FIG. 7, and the similarity feature value calculation operation of the third layer 400 will be described in detail with reference to FIG. 12.

FIG. 5 shows the detailed structure of the first layer in the hypotension prediction model according to an embodiment of the present disclosure.

The hypotension prediction model according to an embodiment of the present disclosure is trained to low-pass filter arterial blood pressure data for training using a training parameter ¢.

Referring to FIG. 5, a first domain θ(x) 501 of the first layer may be composed of a plurality of convolutions with softplus as an activation function and a max pooling layer. Additionally, a second domain δ(x) 502 may include a plurality of dense layers having activation functions of relu and sigmoid.

FIGS. 6A and 6B show the detailed structure of the second layer in the hypotension prediction model according to an embodiment of the present disclosure.

The hypotension prediction model according to an embodiment of the present disclosure is trained to reflect weights on local importance using a training parameter u. Additionally, training can be performed using a training parameter p.

Referring to FIG. 6A, a first domain μ(x) of the second layer may include at least one convolution with softplus as an activation function, at least one max pooling layer, and a plurality of dense layers having activation functions of relu and sigmoid.

In addition, referring to FIG. 6B, a second domain ρ(x) of the second layer processes tensors to calculate a weight for each interval and generates a distribution based on given importance and standard deviation.

FIG. 7 shows the detailed structure of the third layer in the hypotension prediction model according to an embodiment of the present disclosure.

The hypotension prediction model according to an embodiment of the present disclosure is trained to perform probability inference based on a logistic regression layer using training parameters τ and β.

Referring to FIG. 7, a first domain τ(x) 701 of the third layer processes importance tensors throughout shapelet data similarity tensors and selects an interval having the maximum similarity.

Additionally, a second domain β(x) 702 may include at least one dense layer having an activation function of sigmoid.

FIGS. 8 and 9 are diagrams illustrating the process of performing wavelet transform on arterial blood pressure data for training according to an embodiment of the present disclosure.

The hypotension prediction model learning method according to an embodiment of the present disclosure compresses time-series data through low-pass filtering using wavelet transform, thereby leaving the overall signal trend.

First, as shown in FIG. 8, time series compression is performed from the initial ABP input value. Here, a frequency cutoff coefficient in the formula of the low-pass filter used for time series compression is learned as a parameter learned in the model.

According to an embodiment of the present disclosure, time series compression is performed by convolution of a low-pass filter, and window intervals are set to overlap for compressed ABP data (sliding window), thereby expanding the dimension. For example, as shown in FIG. 9, compressed ABP data 900 is divided into local intervals with a frame size of 50, and the compressed ABP data can be dimensionally expanded by allowing some intervals of the window to overlap (901, 902, 903, 904, 905). Although the frame size is exemplified in an embodiment of the present disclosure, the present disclosure is not limited thereto.

FIG. 10 is a diagram illustrating the process of generating shapelet data according to an embodiment of the present disclosure.

The apparatus 10 for predicting hypotension according to an embodiment of the present disclosure generates each piece of trend data as shapelet data depending on the shape.

Here, shapelet data means the shape (local shape) of a specific interval within time series which is regarded as a characteristic.

According to an embodiment of the present disclosure, a plurality of pieces of trend data is learned as 30 pieces of shapelet data and similarities between all local intervals of the compressed time series and the shapelet data are calculated.

FIG. 11 is a diagram illustrating a training process for assigning a weight to a training interval required for hypotension prediction according to an embodiment of the present disclosure.

The apparatus 10 for predicting hypotension according to an embodiment of the present disclosure is trained to select a training interval required for hypotension prediction among a plurality of intervals and assign a weight thereto.

Specifically, training is performed such that five intervals 1101, 1102, 1103, 1104, and 1105 that have served to predict hypotension among all local intervals 1100 of a compressed time series are weighted and set as the average value of Gaussian distributions, and the probability distribution obtained by adding all five Gaussian distributions formed from the five local intervals 1101, 1102, 1103, 1104 and 1105 can be used as a weight in the compressed time series.

Although five intervals are described as an example in one embodiment of the present disclosure, but the present disclosure is not limited thereto.

FIG. 12 is a diagram illustrating a training process for calculating similarity feature values between shapelet data of a training interval and the trend data according to an embodiment of the present disclosure.

The apparatus 10 for predicting hypotension according to an embodiment of the present disclosure is trained to calculate similarity feature values between shapelet data of a training interval and the trend data on the basis of learned weights.

Here, after calculating similarities 1201 between all local intervals and shapelet data, the highest similarity can be calculated as a feature value 1202 on the basis of each piece of shapelet data.

Specifically, similarities between the local intervals and the model are multiplied by a weight for each interval, and only the value indicating the highest similarity among the local intervals is selected.

Thereafter, a logistic regression having “X” as similarity values considering weights and “y” as the probability of hypotension can be performed.

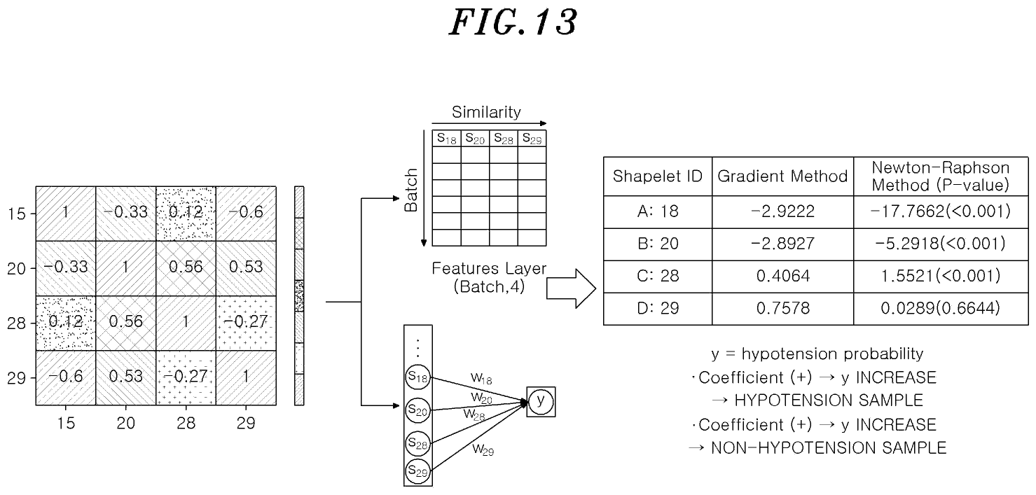

FIG. 13 is a diagram showing final selection of statistically significant shapelets among shapelets learned in the method for predicting hypotension according to an embodiment of the present disclosure through logistic regression coefficient analysis.

FIG. 13 shows the results of selecting local models having correlations of 0.8 or less among similarity values with respect to 30 local models, and it can be ascertained that coefficients of logistic regression performed with the similarity values of the selected local models are calculated using two methods to show generalization of results.

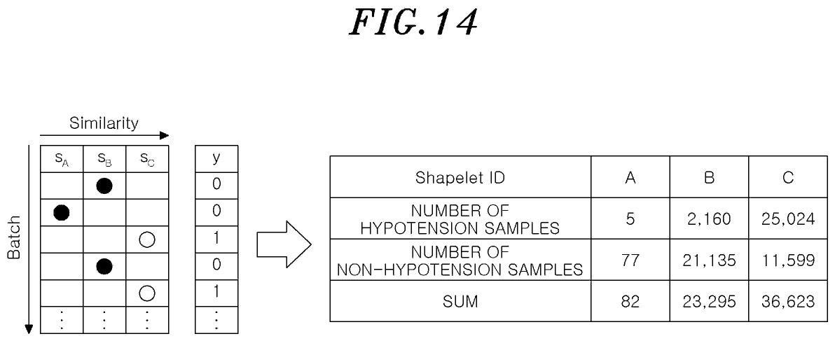

FIG. 14 is a diagram illustrating searching for samples having, as a maximum similarity, each of shapelets selected in the method for predicting hypotension according to an embodiment of the present disclosure, and totaling the number of samples in each of a case of hypotension and a case of non-hypotension.

Referring to FIG. 14, among three pieces of shapelet data for each sample, shapelet data having the maximum similarity is found, and target values of samples showing a high similarity with each shapelet data can be checked.

Here, the presence or absence of hypotension is expressed as y=0/1, and the number can be counted.

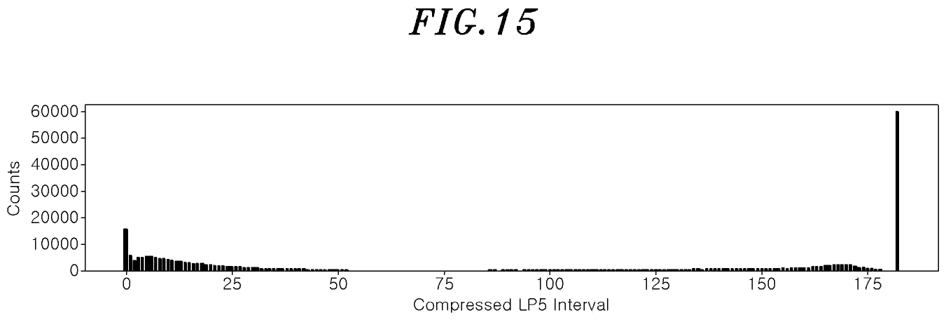

FIG. 15 is a graph showing an aggregate for each weighted point in the entire compressed interval in the method for predicting hypotension according to an embodiment of the present disclosure.

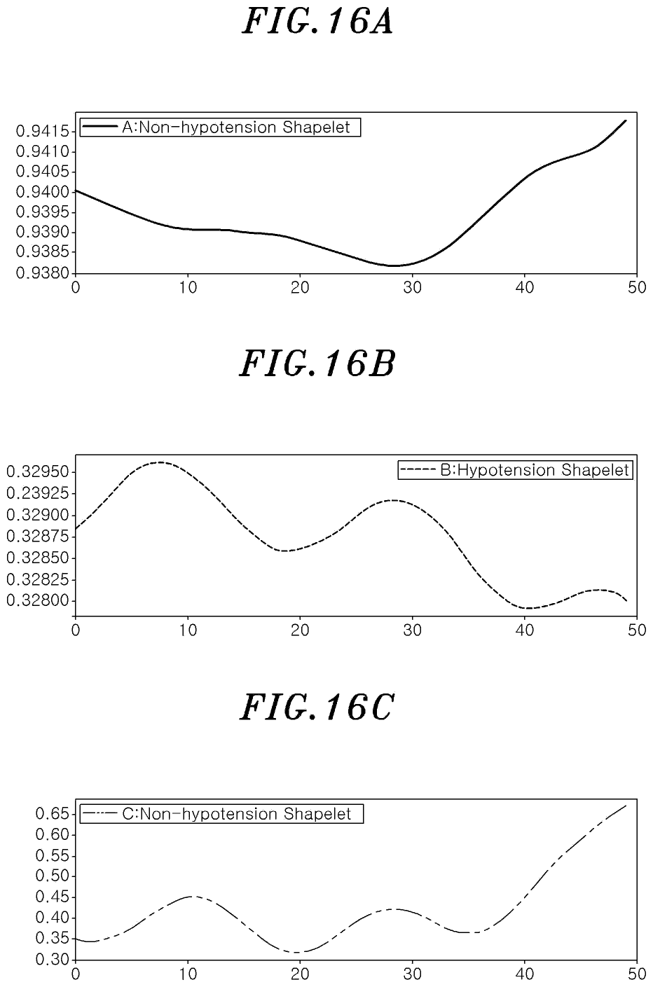

FIGS. 16A to 16C are graphs showing visualization of shapelets selected using the method for predicting hypotension according to an embodiment of the present disclosure.

FIG. 16A shows shapelet data similar to a hypotension sample.

FIG. 16B and FIG. 16C show shapelet data similar to non-hypotension samples.

Accordingly, it is possible to intuitively interpret the variability of ABP waveform low-frequency components or the overall blood pressure trend change by visualizing the shape of shapelet data, and it can be ascertained that there is a clear difference in shapelet data shape and coefficient between hypotension samples and non-hypotension samples.

In addition, a recording medium storing a computer program including instructions for performing a hypotension prediction method including receiving arterial blood pressure data of a subject and determining whether the subject has hypotension using a hypotension prediction model on the basis of changes in each measurement interval of the arterial blood pressure data may be provided.

In addition, a computer program stored in a computer-readable storage medium and including instructions for performing a hypotension prediction method including receiving arterial blood pressure data of a subject and determining whether the subject has hypotension using a hypotension prediction model on the basis of changes in each measurement interval of the arterial blood pressure data may be provided.

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable storage medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

What is claimed is:

1. An apparatus for predicting hypotension of a subject, comprising:
a memory configured to store one or more instructions and a pre-trained hypotension prediction model; and
a processor configured to execute the one or more instructions stored in the memory, wherein the instructions, when executed by the processor, cause the processor to:
determine an arterial blood pressure data of the subject,
input the arterial blood pressure data of the subject into the hypotension prediction model, and
determine whether the subject has hypotension using an output result of the hypotension prediction model,
wherein the hypotension prediction model is trained to determine whether a training subject has hypotension for a training arterial blood pressure data of the training subject.

2. The apparatus of claim 1, wherein the hypotension prediction model is trained by a training input data including a plurality of intervals of the training arterial blood pressure data of the training subject and a label data including whether or not hypotension has occurred for each of the plurality of intervals of the training arterial blood pressure data.

3. The apparatus of claim 2, wherein the hypotension prediction model includes:
a first layer trained to extract trend data for each of the plurality of intervals of the training arterial blood pressure data of the training subject by wavelet-transforming the plurality of intervals of the training arterial blood pressure data of the training subject; and
a shapelet data generation module configured to generate a shapelet data corresponding to the trend data.

4. The apparatus of claim 3, wherein the hypotension prediction model is trained to low-pass filter the training arterial blood pressure data using training parameters trained in the first layer.

5. The apparatus of claim 3, wherein the hypotension prediction model includes:
a second layer trained to assign a weight to at least one of intervals among the plurality of intervals of the training arterial blood pressure data of the training subject; and
a third layer trained to calculate similarity feature value between shapelet data of the at least one interval and the trend data on the basis of the assigned weight.

6. The apparatus of claim 3, wherein the processor is configured to input the arterial blood pressure data of the subject to the hypotension prediction model, and to calculate similarity between trend data for each of the plurality of intervals of the arterial blood pressure data of the subject and the shapelet data generated by the shapelet data generation module.

7. The apparatus of claim 6, wherein the processor is configured to calculate hypotension probability for the calculated similarity of the trend data for each of the plurality of intervals of the arterial blood pressure data of the subject using a logistic regression layer.

8. A method for training a hypotension prediction model performed by an electric device including a processor, comprising:
preparing a training data including a training input data and a label data, wherein the training input data includes a plurality of intervals of the training arterial blood pressure data of a training subject and, the label data includes whether or not hypotension has occurred for each of the plurality of intervals of the training arterial blood pressure data of the training subject;
inputting the training input data into the hypotension prediction model;
training the hypotension prediction model to extract trend data for each of the plurality of intervals of the training arterial blood pressure data of the training subject by wavelet-transforming the plurality of intervals of the training arterial blood pressure data of the training subject; and
generating a shapelet data corresponding to the trend data.

9. The method of claim 8, wherein the training of the hypotension prediction model to extract trend data includes updating training parameters trained in a first layer such that the hypotension prediction model low-pass filters the training arterial blood pressure data of the training subject.

10. The method of claim 8, further comprising:
training the hypotension prediction model to assign a weight to at least one of intervals among the plurality of intervals of the training arterial blood pressure data of the training subject; and
training the hypotension prediction model to calculate similarity feature values between shapelet data of the at least one interval and the trend data on the basis of the assigned weight.

11. A method for predicting hypotension performed by an apparatus for predicting hypotension of a subject, the method comprising:
preparing a pre-trained hypotension prediction model;
determining an arterial blood pressure data of the subject;
inputting the arterial blood pressure data of the subject into the hypotension prediction model, and
determining whether the subject has hypotension using an output result of a hypotension prediction model,
wherein the hypotension prediction model is pre-trained to determine whether a training subject has hypotension for a training arterial blood pressure data of the training subject.

12. The method of claim 11, wherein the hypotension prediction model is trained by a training input data including a plurality of intervals of the training arterial blood pressure data of the training subject and a label data including whether or not hypotension has occurred for each of the plurality of intervals of the training arterial blood pressure data.

13. The method of claim 12, wherein the hypotension prediction model includes:
a first layer trained to extract trend data for each of the plurality of intervals of the training arterial blood pressure data of the training subject by a wavelet-transforming the plurality of intervals of the training arterial blood pressure data of the training subject; and
a shapelet data generation module configured to generate a shapelet data corresponding to the trend data.

14. The method of claim 13, wherein the determining of whether the subject has hypotension includes inputting the arterial blood pressure data of the subject to the hypotension prediction model, and calculating similarity between the trend data for each of the plurality of intervals of the arterial blood pressure data of the subject and the shapelet data generated by the shapelet data generation module.

15. The method of claim 14, wherein the determining of whether the subject has hypotension includes calculating hypotension probability for the calculated similarity of the trend data for each of the plurality of intervals of the arterial blood pressure data of the subject using a logistic regression layer.
